(19) United States
(12) Patent Application Publication
(10) Pub. No.: US 2019/0198163 A1
(43) Pub. Date: Jun. 27, 2019

(54) A SYSTEM AND METHOD FOR OPTIMISING SUPPLY NETWORKS

(71) Applicant: Babylon Partners Limited, London (GB)

(72) Inventors: Ali PARSA, London (GB); Gary MUDIE, London (GB); Prem SHARMA, London (GB)

(21) Appl. No.: 16/325,636

(22) PCT Filed: Aug. 11, 2017
(86) PCT No.: PCT/GB2017/000121

§ 371 Date: Feb. 14, 2019

(30) Foreign Application Priority Data
Aug. 15, 2016 (GB) GB1613954.5

(51) Int. Cl. G16H 40/20; G06F 16/2455

(52) U.S. Cl. CPC G16H 40/20; G06F 16/2455

(57) ABSTRACT

A system and method of optimising supply networks comprises the method steps of i) receiving a resource request from a user; ii) assessing the requirements of the request; iii) identifying all potential resources within a database that would fulfil the requirements; iv) ranking the potential resources in order of preference; v) allocating the most preferable resource to fulfil the request; and wherein, in the step of identifying all potential resources that would fulfil the requirements, all resource that does not fulfil primary requirements is discarded.

### FIELD OF THE INVENTION

The present invention relates to a system for optimising supply networks. More particularly, the present invention relates to a system for optimising supply networks for medical suppliers. The present invention also relates to a method for optimising supply networks. More particularly, the present invention relates to a method for optimising supply networks for medical suppliers.

### BACKGROUND

It can be increasingly difficult, for a number of reasons, for healthcare providers to provide an appointment for a patient so that they can see a qualified professional such as a doctor in a timely fashion. This is partly due to population increases and similar, which are negatively impacting on the ratios of patients to qualified professionals in any given geographic area, and partly due to changes in demographics, where patients may need to see a qualified professional who also meets cultural requirements such as gender or language. Furthermore, the numbers of patients which a doctor or other professional is expected to deal with has also increased, increasing their workload. At present, it is usual for patients and doctors to nearly always be geographically generally co-located so that patients will be able to have a physical appointment with a doctor or other healthcare professional, with limited travelling required. However, populations are less and less homogenous, and it is common for individuals and families to travel to other countries of areas for work or similar. In these circumstances, language and cultural barriers can increase the difficulties of providing a diagnosis and recommending treatment.

There are a number of other services that are also at present usually provided face-to-face, such as for example mortgage advice, account managers or similar. Provision of these services can also encounter similar problems, with a small number of professionals available in a particular area, and a large (and growing) customer base that requires servicing.

It is an object of the present invention to provide a system for optimising supply networks which goes some way to overcoming the abovementioned disadvantages or which at least provides the public or industry with a useful choice.

It is a further object of the invention to provide a method for optimising supply networks which goes some way to overcoming the abovementioned disadvantages or which at least provides the public or industry with a useful choice.

Further objects and advantages of the invention will be brought out in the following portions of the specification, wherein the detailed description is for the purpose of fully disclosing the preferred embodiment of the invention without placing limitations thereon.

The background discussion (including any potential prior art) is not to be taken as an admission of the common general knowledge.

### SUMMARY OF THE INVENTION

The term “comprising” as used in this specification and indicative independent claims means “consisting at least in part of”. When interpreting each statement in this specification and indicative independent claims that includes the term “comprising”, features other than that or those prefaced by the term may also be present. Related terms such as “comprise” and “comprises” are to be interpreted in the same manner.

As used herein the term “and/or” means “and” or “or”, or both.

As used herein “(s)” following a noun means the plural and/or singular forms of the noun.

In an aspect, the invention may broadly be said to consist in a method of optimising supply networks, comprising the steps of;
i) receiving a resource request from a user;
ii) assessing the requirements of the request;
iii) identifying all potential resources within a database that would fulfil the requirements;
iv) ranking the potential resources in order of preference;
v) allocating the most preferable resource to fulfil the request;
wherein, in the step of identifying all potential resources that would fulfil the requirements, all resource that does not fulfil primary requirements is discarded.

In an embodiment, all resource that does not fulfil a set of pre-configured primary requirements is discarded by specifying the primary requirements in an initial database query.

In an embodiment, all resource that does not fulfil a set of pre-configured primary requirements is discarded by applying filtering rules to a set of initially identified potential resource.

In an embodiment, the primary requirements comprise one or more of: language compatibility; expertise, contractual availability.

In an embodiment, in the step of allocating the most preferable resource to fulfil the request, if two or more resources are substantially equally capable of fulfilling the request, the resource with less future demand is allocated.

In an embodiment, in the step of allocating the most preferable resource to fulfil the request, if no suitable resource is available, the suitable resource that is next available is allocated.

In an embodiment, in the step of ranking the potential resource in order of preference, the request is further assessed by one or both of a set of user preferences and a set of management preferences.

In an embodiment, the set of user preferences comprises one or more of: gender preference; time preference; superiority of expertise; format preference.

In an embodiment, the set of management preferences comprises one or more of: effective time utilisation; resource prioritisation; expertise overspill; capacity overspill; contingency timing.

In an embodiment, in the step of ranking the potential resource in order of preference, the request is further assessed by secondary factors that comprise one or more of: symptom type; user history.

In an embodiment, in the step of identifying all potential resource that would fulfil the requirements, a database is interrogated as part of an SQL Query.

In an embodiment, in the step of ranking the potential resources in order of preference, compatible values are calculated for one or more preference factors comprising: time closeness score; busyness score; medical expertise score; overspill cost; unused capabilities score.

In an embodiment, the compatible values comprise numerical values.

In an embodiment, the numerical values of the preference factors are aggregated to provide an overall optimum priority for each potential resource.

In an embodiment, in the step of allocating the most preferable resource to fulfil the request at least the highest-scoring option is allocated to fulfil the request.

In an embodiment, in the step of allocating the most preferable resource to fulfil the request a plurality of the highest-scoring options are presented to the user, the method comprising the additional step of the user choosing which option should be allocated to fulfil the request.

In an embodiment, the method comprises a first initial step of grouping individual resource into supply networks within the database, the individual entries in a supply network sharing one or more resource characteristic.

In an embodiment, the method comprises a second initial step of grouping multiple users into consumer networks within the database, the individual users within a consumer network sharing one or more attributes.

In an embodiment, the method further comprises the step of assessing the attributes of the user between the step of receiving a resource request from a user and assessing the requirements of the request.

In an embodiment, the method comprises a computer-implemented method.

In a second aspect the invention may broadly be said to consist in a system for optimising supply networks comprising a computing system capable of carrying out the method steps of any one of the preceding statements.

In an embodiment, the system comprises control and storage hardware configured to act as a database component and a central controller, and; communication hardware configured to communicate externally to the system to receive user requests.

### BRIEF DESCRIPTION OF THE DRAWINGS

Further aspects of the invention will become apparent from the following description which is given by way of example only and with reference to the accompanying drawings which show embodiments of the device by way of example, and in which:

FIG. 1 shows a schematic overview of a number of customer groups or networks and a number of supply groups or networks, the potential relationship paths between them, and a centralised management and monitoring system that oversees and administers allocation of resource, the potential relationship paths passing through the centralised management and monitoring system that tracks supply and demand and allocates resource accordingly within the overall network.

FIG. 2 shows a schematic view of the components and connections between the centralised management and monitoring system and a database and user terminals.

FIG. 3 shows an example of the method in use, a user logging on to the network to make an appointment, the system assessing the background and existing status of the patient and providing options for a list of clinicians to provide treatment.

FIG. 4 shows a particular relationship path formed between the user and the clinician of the example of FIG. 3, the path linking from the patient as an individual within their customer network, through the centralised management and monitoring system, to an individual clinician with a supply group.

FIG. 5a shows an example of the number of possible connections between patients, and possible contractual obligations or relationships.

FIG. 5b shows the simplification and reduction of the number of possible connections when these are routed through a supplier organisation/supply network.

### DETAILED DESCRIPTION OF THE INVENTION

Embodiments and variations thereof of the present invention will now be described with reference to the figures.

Overview

The present invention provides a system and an associated method for forming a network between a centralised control system, and a number of supplier organisations and customer organisations, and optimising the supply of services within the network as required, with resource requests centrally routed through the centralised control system for maximum efficiency.

The present invention comprises a computer-network-implemented method, and a system of implementing the method, that optimises the supply of services within a network. The invention can be generally referred to as a ‘dynamic supply allocator’. In the embodiment described below the invention is used to optimise the allocation of staff resources for providing remote healthcare consultations within a healthcare network. However, it should be noted that the system and method of the present invention could also be used to optimise the provisioning of any service, especially those where geographic distance is essentially irrelevant or at least less of a limiting factor than may previously have been the case. For example, any global company that has a large pool of services (either centralised or distributed), that could be allocated to a consumer base that is also geographically distributed. For example, the system/method could be used to allocate a financial adviser such as a mortgage advisor or an account manager for financial services, or it could be used to find a commercial agent for an actor, or similar.

The dynamic supply allocator of the present invention involves a network formed from four main elements:
1. Groups of customers/patients, or customer organisations/customer networks 3. These could be for example groups of individual patients 100 who have access as employees of a member organisation 3, or access as individuals.
2. Suppliers of medical services, or supplier organisation/supply networks 2. These are suppliers of services that are grouped by resource characteristic, for example clinicians that provide overnight cover. It should be noted that any resource unit could be entered or listed in multiple groups. For example, a specialist paediatrician could also provide night cover, and would be therefore be listed separately in each of these two separate groups.
3. The potential relationship path/paths 4 between customers/patients and the services they are able to receive. The nature of these relationship paths will be determined by the commercial model under which the patient receives their healthcare. That is, patients who are members of a particular network or subscribers to a particular plan can receive healthcare from clinicians in a particular supply network.
4. A centralised management and monitoring system 1 that oversees and administers allocation of resource (e.g. clinicians) to patients, based on balancing various overlapping and/or conflicting criteria.

It should be noted that the consumer networks 3 and the supply networks 2 are not significant entities on their own. They act as a grouping for patients 100 and clinicians respectively but the significant properties are actually embodied in the contractual relationship between any particular consumer networks and any particular supply network.

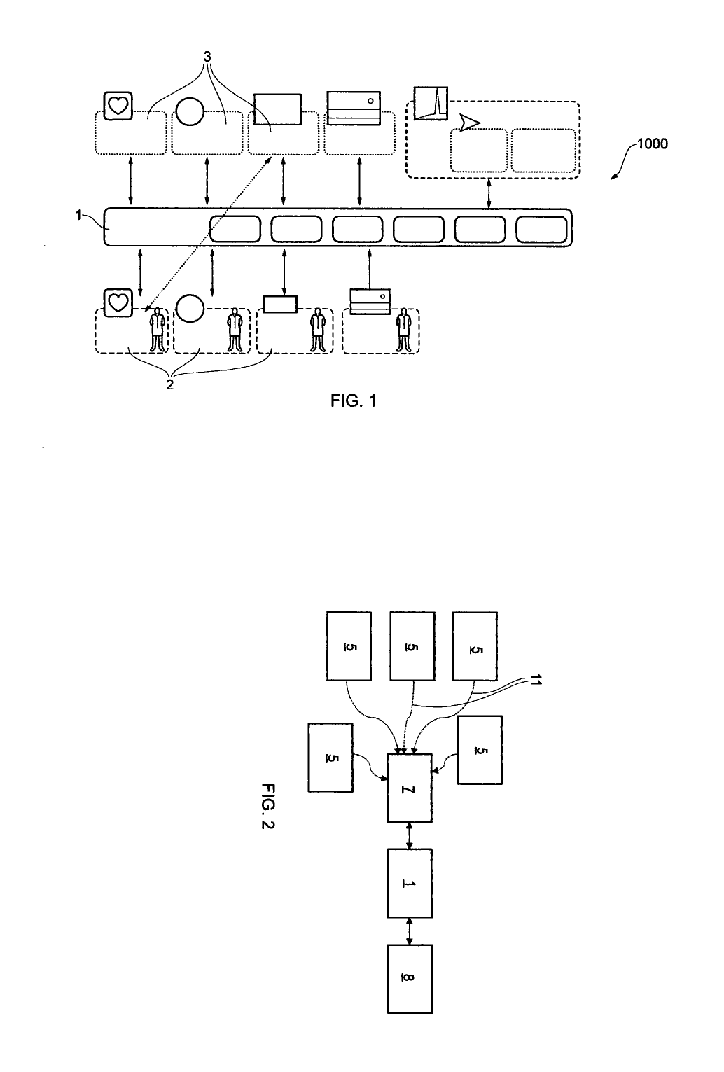

A schematic overview of the system of this embodiment is shown in FIG. 1. The system comprises a network 1000 formed from three main elements: a centralised management and monitoring system 1 (control system 1), suppliers of medical services (supplier organisations/supply networks 2), and customers of those services (customer organisations/customer networks 3). Together, these form a distributed global network of patients and practitioners within a clinical setting, the network formed from potential relationship paths 4 between the supply networks 2 and the customer networks 3 that pass through the control system 1, the control system 1 using the relationship paths 4 as required in order to allocate resources to fulfil demands.

Supplier Networks

The supplier networks 2 comprise loose organisations of different suppliers, the members of any organisation classified based on different properties. For example a supplier organisation may be classified by one or more of: specialism, location, time zone, or it's ability to provide a particular service to a specific group of consumers (a particular service would be for example ‘health insurance’).

An example would be those suppliers who are able to provide an out-of-hours service, who are formed into a loose supplier network.

Another example might include a GP Clinician Network in the UK that forms one network. The NHS network in the UK could form another group. Surgeons across the EU with a particular specialty could form another group.

The central control system 1 comprises or has access to a database that is populated with attributes/properties of the suppliers within the supplier networks 2. These attributes/properties include all relevant information, such as for example:
Language(s) spoken.
Quantified medical capabilities/expertise (this could be done through a confidence mapping questionnaire or extracted from a national register of training levels depending on the country in which the clinician resides).
Gender.

A number of other attributes may also be allocated to the suppliers, for example as follows:
Pre-negotiated and defined business rules that specify the scope of the service that can be provided. That is, a supplier may be capable or qualified to provide a particular service, but may be limited in their ability to do so due to pre-existing (pre-negotiated and defined) limitations. These could include for example:
   Limited operating hours.
   Additional cost per patient/appointment.
   Specific medical criteria under which services may be provided (e.g. only able to supply services for patients with a particular disease or exhibiting particular symptoms).
   Specific ‘overspill’ criteria under which services may be provided (e.g. no more than 5 patients per hour).

Customer Networks

Each customer network 3 is comprised of a group of consumers/users/patients 100. Within any particular group or sub-grouping, the patients 100 have the same list of attributes and will follow the same business rules within the central control system 1. Examples of the classification attributes can be: country location, insurance type, affiliate-based, spoken and preferred languages, age, gender, etc.

Centralised Control System

The centralised control system 1, or centralised control platform 1 (the dynamic supply allocator) comprises a dynamic demand and supply scheduling algorithm for the network. This algorithm runs on any suitable apparatus or network, such as for example a centralised or distributed server system. The apparatus/network also comprises or has access to a communication means 7 and a database component 8. As shown in FIG. 2, the communication means 7 allows the centralised control platform 1 to receive requests from the users via their terminals 5, and to send messages to allocate resource as required. The communication means 7 also allows updating or populating of the databases 8. The communication means 7 will be one or more of a hardwired landline, a wireless transmitter/receiver, a mobile telephone network, or any other suitable communication device and method.

The key purpose of the centralised control platform 1 is to optimise the supply of services from the supplier networks 2 to the customer networks 3, based on real-time demand, in order to serve consumer needs in the most efficient manner possible.

Operation

The key activity flow can be generalised as follows:

A patient 100 within a customer network 3 will make a request for medical services. The request is made via a user terminal 5 such as for example a laptop, desktop, tablet, mobile device, or similar, which is loaded with the appropriate software, such as for example an app on a mobile device. The request can be entered directly from the user's terminal 5, or via an intermediary such as a receptionist or operator or similar, who will receive the user's request verbally (in person or by telephone), and enter the details into their own terminal. The terminals 5 are in contact with the hardware system on which the central control system 1 software is operating. For example, the central control system 1 could be operating on a distributed or centralised server network, and the user requests are communicated via any suitable communication means, such as for example wireless, hardwired lines, a mobile telephone network, or any combination of these or similar communication networks (the communication paths/routes shown generally in FIG. 2 by dotted lines 11).

The time at which the request is made for (i.e. the time required for the appointment—e.g. 8.00 AM the following morning) is recorded and communicated to the central control system 1. This is one of the primary factors that decides the particular relationship path 40 between the supply networks 2, and the patient or user. The patient or user can add or specify further optional criteria or preferences (secondary preferences) to the request (e.g. preferred gender of clinician). Once all of the criteria are received, the central control system 1 processes the request, allocating a particular resource to the patient for a particular time slot. The patient and clinician then hold the consultation and address any healthcare needs appropriately.

The central control system 1 is involved only in the first step—that of allocating a patient to a particular clinician at a particular time. Once the resource has been allocated to a particular time slot, the central control system 1 plays no further role in the request or the actual healthcare service provisioning and any follow-up activities such as prescriptions, blood tests, etc.

When allocating a clinician to a patient multiple factors influence which clinician should be chosen. In this embodiment, these are grouped into three main categories: ‘Strict Requirements/Primary Requirements’; ‘Patient Preferences’ and; ‘Management Preferences’.

Examples of the requirements and preferences for each category are outlined below. These should be considered to be inclusive, and the requirements and preferences should not be taken as limited to these examples:
Strict Requirements/Primary Requirements
   Language Compatibility: Patients and clinicians must speak the same language.
   Expertise: Clinicians must be able to provide the requested medical care (i.e. Have a suitable level of experience in the disease area).
   Contractual Availability; Contractual relationships must allow that clinician to service requests from that patient (for example not allocating an out-of-hours clinician to a patient whose commercial model only includes business-hours cover). That is, allowability within the bounds of existing pre-set relationships.
User Preferences
   Time Preference: Patients specify a desired time and should get an appointment as close as possible to their requested time.
   Gender Preference: Some patients may prefer a particular gender of clinician but might be willing to see a different gender of clinician if it allows them to see a clinician sooner.
   Superiority of Expertise: A patient should be allocated to a clinician with superior expertise in the requested medical area (but will always be allocated to a clinician with sufficient expertise).
   Format Preference: Preferred appointment format (Face-to-face, video, audio).
Management Preferences
   Effective time utilisation: Clinicians should not be under-utilised and waiting for patients.
   Prioritisation of standard resource: Clinicians with unusual capabilities (e.g. the ability to speak Japanese) should not be consumed unnecessarily as it increases risk of being unable to fulfil a later request (e.g. if a bilingual clinician was allocated to an English speaking patient when other monoglot English speaking clinicians were available, a later request from a monoglot Japanese speaking patient could not be fulfilled). That is, standard resource (e.g. clinicians who are monolingual or only have the standard skillset) is prioritised over resource with the standard skillset and also secondary advantages, all other factors being substantially the same.
   Expertise Overspill: Clinical scope may be defined in the contract, e.g. a specialist hospital such as Great Ormond Street Hospital (a specialist children's hospital) may offer to service requests from any patient regardless of which consumer network or networks they are members of, if the request relates to an area in which they have expertise, such as for example childhood meningitis.
   Capacity Overspill: Capacity management rules, Service Level Agreements and additional charges specified in contracts (e.g. if a particular supply network normally only services requests from a particular consumer network, but has agreed to act as ‘overspill’ capacity management for other consumer networks at a fee per patient/consultation).
   Contingency Timing: Clinicians do not want to be fully booked as they have no contingency time in case consultations overrun and delay their overall schedule.

Other factors that can be taken into consideration could be as follows, and generally relate to an initial identification of the current medical need:
Disease area/symptom type.
History (e.g. has this patient had previous appointments with the same or similar symptoms, or have they recently been allocated resource for consultation for similar or potentially related issues).

Once the data is collected (the primary requirements, the preferences, and any other factors), the central control system 1 optimises the allocation of clinical services, fulfilling the primary requirements, and balancing between the overlapping and/or conflicting secondary preferences and factors, in order to produce a particular relationship path 40 that allocates resource to fulfil the request.

The central control system 1 performs the following broad steps:
1. Identifying a list of ALL clinicians and appointment slots that could service that patient. In the preferred embodiment, this is accomplished by joining the relevant database tables in the database 8 as part of a SQL Query.
2. Discarding invalid options that do not meet the strict requirements. This is accomplished by specifying criteria in the initial database query, or by applying row-by-row filtering rules after extracting the initial list from the database.
3. Computing numerical values for each of multiple ‘preference factors’:
   “Time Closeness Score” (requested time compared to actual/current time)
   “Busyness Score” (calculated on a factor of slots booked an available over the three hours following the request).
   “Medical Expertise Score” (from the suppliers records)
   “Overspill Cost” (is there a defined penalty fee for exceeding any specified limits in the Service Level Agreement?)
   “Unused Capabilities Score” (a weighted value of skills held by that clinician that are not being used)
4. Applying a ‘weighting factor’ to each of the ‘preference factors’ (each preference factor is in a different format minutes, percentage busyness, cost etc. Different factors have more or less significance than others, and so need to be weighted differently).
5. Aggregating the weighted preference factors to give an overall optimum priority for each option of a clinician and time slot
6. Either informing the patient of the allocated clinician that meets all their requested criteria (the highest-scoring option), or providing options and asking the patient/user to make a choice.

### Example

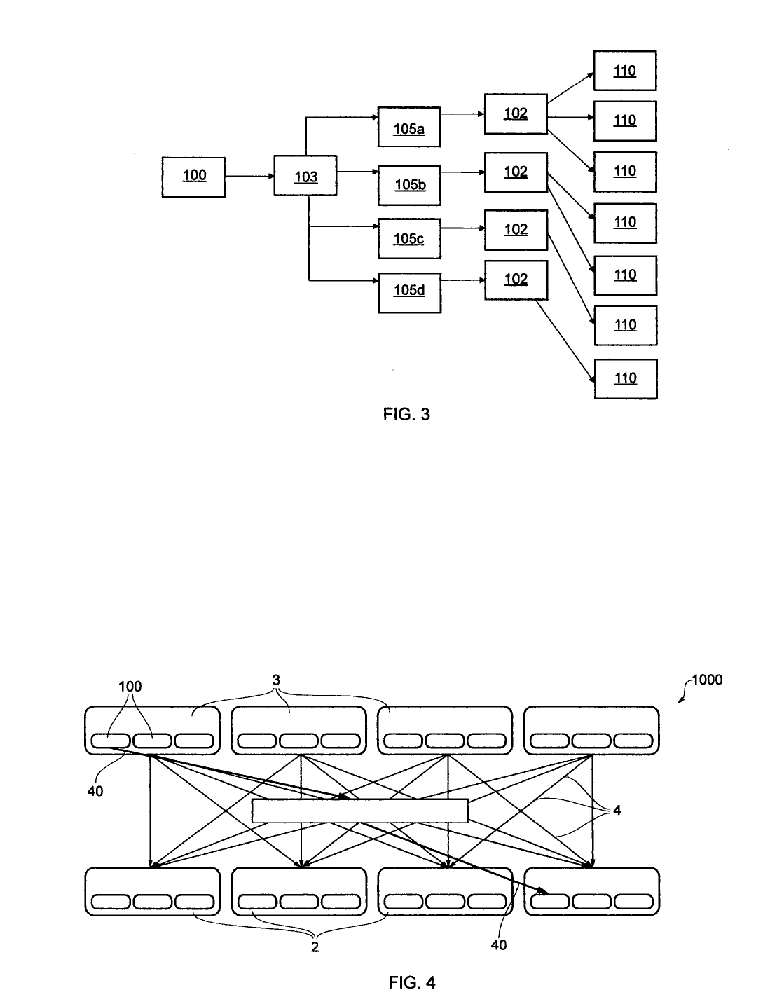

An example of how this works in use is outlined in detail below, with reference to FIG. 3, and tables 1, 2 and 3 in Appendix A:

A patient 100 logs into the system 1 via their terminal to make an appointment. They are already registered as part of a customer network 103 (the customer network 103 could be for example the ‘mothercare’ network in the UK), so their attributes are stored/listed on the database 8, and can be accessed by the central control system 1. The patient 100 is listed as speaking English, and is requesting an appointment for themselves on a specific date at a specific time (e.g. 1 Aug. 2017 at 10 PM) regarding back pain. The patient 100 is linked to four supply networks 102 through pre-existing contractual relationships 105a, 105b, 105c, and 105d, each of which contains specific criteria.

The central control system 1 receives the request, and interrogates the database 8, accessing their profile and pre-registered attributes. The central control system 1 then carries out the following steps:
Step 1: identify all clinicians 110 that could theoretically provide services to that patient (the list for example comprises Dr Takagashi, Dr Anderson, Dr Brown, Dr Carter, Dr Darwin, Dr Eureka and Dr Famham).
Step 2: discard all the invalid options (Dr Takagashi does not speak English. The contractual relationship with Great Ormond Street Hospital only covers children with suspected meningitis so Dr Farnham is discounted).
Step 3: compute numerical values for each of the preference factors shown in Table 1 in Appendix A.

As shown in table 1, ‘Busyness Score’ is a percentage, ‘Time closeness score’ is in minutes, ‘Medical Expertise Score’ is given a score out of ten, and the ‘unused capabilities score’ is measured out of one hundred and seventy (the maximum value of all the weighted capabilities that can be underutilised, such as for example spoken languages, sign language or a given specialism like managing drug addicts).
Step 4: apply a weighting factor to these scores so they can be directly related.
Step 5: combine the scores into a single score.

The weighting factors are configurable, allowing the algorithm of the central control system 1 to be fine-tuned. The weighting factors are also different for each consumer network 3, as different commercial models give different priorities to each preference factor. For example, the weighting factor for “Time Closeness Score” in table 2 as shown in Appendix A includes an exponent, as there is a non-linear relationship between the time delay for a patient and the impact it will have and therefore how disruptive it would be to have a later appointment.

In this example the totalling function is carried out in order to sum the weighted values, but a different calculation could also be used.

Table 3 as shown in Appendix A completes the worked example and shows the total scores, showing that the appointment with Dr Carter at the time requested is the best match for this clinical services request.

It can be seen that the system and method of the present invention solves the problem of efficiently and quickly allocating the most appropriate resource to meet a request for a resource from a pool of many different possibilities. In the example of allocating medical resource for digital healthcare consultations the geographic location of the patient and clinician is irrelevant, and therefore the pool of clinicians that can be chosen from is not limited by population density and there could be many thousands of potential matches.

By allocating the most appropriate resource to meet a request for a service three factors are optimised:
1. The service request is fulfilled to the best possible standard
2. The strain of demands on service suppliers is reduced as much as possible
3. The ability to service different request types is kept as high as possible

A number of other factors are also applied by the central control system 1 to narrow the search results quickly and effectively:
‘Strict need’/‘Invalid combination’
For example, if the clinician speaks a different language to the patient, the clinician can be eliminated or discarded from the list of potential providers immediately. Similarly, if the patient has requested a female clinician, and so should not be allocated a male clinician, all male clinicians can immediately be eliminated or discarded.
‘Clinician Busyness’/‘Future Demand’
If two or more resources are substantially equally capable of fulfilling the request, the resource with less future demand or fewer future demands is allocated. For example, if two clinicians are capable of being allocated to a patient, if all the other factors are substantially equal the clinician with fewer upcoming appointments should be allocated to this patient.
‘Time delay’
If no clinician is available at the time requested by the patient and two clinicians are available at a later time, the clinician whose availability is closest to the requested time is allocated to this patient.

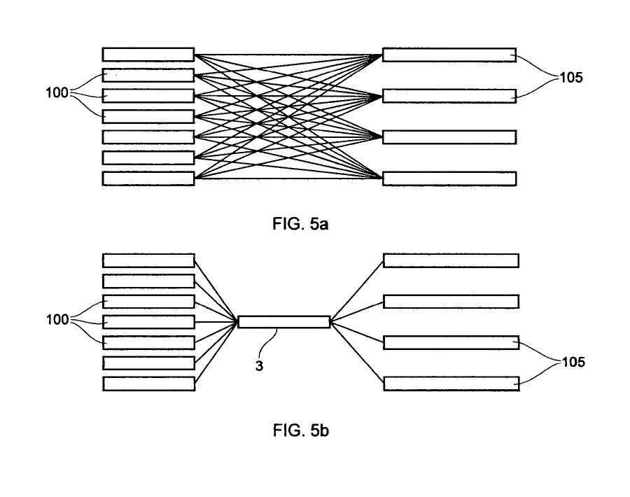

As outlined above, a key component of the embodiment described is the relational database 8 which stores the properties of patients, clinicians and contractual relationships, and which allows connections to be formed between them. Grouping patients into consumer networks and clinicians into supply networks allows for a simplification in the number of possible connections. As shown in FIG. 5a the relationships between seven patients 100 and four contractual relationships 105 rapidly adds up—there are twenty-eight connections between the seven patients 100 and the four contractual relationships 105 (seven times four). However, when a consumer network 3 is added, there are only eleven connections (seven+four). This demonstrates how, even for a small subset, the number of connections increases exponentially as the number of patients and contracts within the supply networks increases. The same benefits in reduced connections is seen with supply networks, where the number of connections between clinicians and contractual relationships is significantly reduced. The impact of reducing the number of connections is a reduction in query execution time at the expense of query syntax complexity.

This greatly assists with reducing the time required to complete a particular query, and to increase the speed with which results are delivered, using less processing power in the system. Further, by having an early or initial step where invalid options that do not meet the strict requirements are discarded, processing power and memory requirements can be reduced significantly.

TABLE 1

| | | | | | | | Unused | |
|---|---|---|---|---|---|---|---|---|
| | | | Busyness | Time closeness | Overspill Limit | Overspill | Capabilities | Medical |
| Clinician | Date/Time | Format | Score | score | Remaining | Cost | Score | Expertise Score |
| Dr Armstrong | 02/06/17 07:00 | Audio | 0/100 | +540 Minutes | NULL | NULL | 12/170 | 8/10 |
| Dr Armstrong | 02/06/17 07:10 | Audio | 0/100 | +550 Minutes | NULL | NULL | 12/170 | 8/10 |
| Dr Brown | 02/06/17 09:00 | Video | 0/100 | +660 Minutes | NULL | NULL | 22/170 | 6/10 |
| Dr Brown | 01/06/17 22:00 | Video | 15/100 | +/−0 Minutes | NULL | NULL | 15/170 | 7/10 |
| Dr Carter | 01/06/17 22:30 | Video | 15/100 | +30 Minutes | NULL | NULL | 15/170 | 7/10 |
| Dr Darwin | 01/06/17 21:50 | Audio | 35/100 | −10 Minutes | NULL | NULL | 40/170 | 9/10 |
| Dr Darwin | 01/06/17 22:20 | Audio | 35/100 | +20 Minutes | NULL | NULL | 40/170 | 9/10 |
| Dr Darwin | 01/06/17 22:30 | Audio | 35/100 | +30 Minutes | NULL | NULL | 40/170 | 9/10 |
| Dr Eureka | 01/06/17 21:50 | Video | 60/100 | −10 Minutes | 27 | £10 | 10/170 | 7/10 |

TABLE 2

| | |
|---|---|
| Format Score | 25 points if format does not match |
| Busyness Score | Value used as points unchanged |
| Time Closeness Score | (Time/6)\hat{ }2.5 |
| Overspill limit remaining | (25 − Value) points |
| Overspill Cost | Value used as points unchanged |
| Unused Capabilities Score | (Value/Maximum)*200 |
| Medical Expertise Score | 50 − (Value*5) |
| Total Function | Sum of weighted values |

TABLE 3

| | | | | Time | | | | Medical | |
|---|---|---|---|---|---|---|---|---|---|
| | | Format | Busyness | closeness | Overspill Limit | Overspill | Unused | Expertise | TOTAL |
| Clinician | Date/Time | Score | Score | score | Remaining | Cost | Capabilities Score | Score | SCORE |
| Dr Carter | 01/06/17 22:00 | 0 | 15 | 0 | 0 | 0 | 18 | 15 | 48 |
| Dr Eureka | 01/06/17 21:50 | 0 | 60 | 0 | 0 | 10 | 12 | 15 | 97 |
| Dr Brown | 01/06/17 22:30 | 0 | 15 | 50 | 0 | 0 | 18 | 15 | 98 |
| Dr Carter | 01/06/17 21:50 | 25 | 35 | 0 | 0 | 0 | 47 | 5 | 112 |
| Dr Darwin | 01/06/17 22:20 | 25 | 35 | 20 | 0 | 0 | 47 | 5 | 132 |
| Dr Darwin | 01/06/17 22:30 | 25 | 35 | 50 | 0 | 0 | 47 | 5 | 162 |
| Dr | 02/06/17 07:00 | 25 | 0 | 76840 | 0 | 0 | 14 | 10 | 76889 |
| Armstrong | | | | | | | | | |
| Dr | 02/06/17 07:10 | 25 | 0 | 80450 | 0 | 0 | 14 | 10 | 80599 |
| Armstrong | | | | | | | | | |
| Dr Brown | 02/06/17 09;00 | 0 | 0 | 126900 | 0 | 0 | 26 | 20 | 126946 |

1. A method of optimizing supply networks, comprising:
i) receiving a resource request from a user;
ii) assessing the requirements of the request;
iii) identifying ail potential resources within a database that would fulfil the requirements;
iv) ranking the potential resources in order of preference;
v) allocating the most preferable resource to fulfil the request;
wherein, in identifying all potential resources that would fulfil the requirements, all resource that does not fulfil primary requirements is discarded.

2. The method of optimizing supply networks of claim 1, wherein all resource that does not fulfil a set of pre-configured primary requirements is discarded by specifying the primary requirements in an initial database query.

3. The method of optimizing supply networks of claim 1, wherein all resource that does not fulfil a set of pre-configured primary requirements is discarded by applying filtering rules to a set of initially identified potential resource.

4. The method of optimizing supply networks of claim 2, wherein the primary requirements comprise one or more of: language compatibility; expertise, contractual availability.

5. The method of optimizing supply networks of claim 1, to wherein in allocating the most preferable resource to fulfil the request, if two or more resources are substantially equally capable of fulfilling the request, the resource with less future demand is allocated.

6. The method of optimizing supply networks of claim 1, wherein in allocating the most preferable resource to fulfil the request, if no suitable resource is available, the suitable resource that is next available is allocated.

7. The method of optimizing supply networks of claim 1, wherein in ranking the potential resource in order of preference, the request is further assessed by one or both of a set of user preferences and a set of management preferences.

8. The method of optimizing supply networks of claim 7, wherein the set of user preferences comprises one or more of: gender preference; time preference; superiority of expertise; format preference.

9. The method of optimizing supply networks of claim 7, wherein the set of management preferences comprises one or more of: effective time utilization; resource prioritization; expertise overspill; capacity overspill; contingency timing.

10. The method of optimizing supply networks of claim 1, wherein in ranking the potential resource in order of preference, the request is further assessed by secondary factors that comprise one or more of: symptom type; user history.

11. The method of optimizing supply networks of claim 1, wherein in identifying all potential resource that would fulfil the requirements, a database is interrogated as part of an SQL Query.

12. The method of optimizing supply networks of claim 7, wherein in ranking the potential resources in order of preference, compatible values are calculated for one or more preference factors comprising: time closeness score; busyness score; medical expertise score; overspill cost; unused capabilities score.

13. The method of optimizing supply networks of claim 12, wherein the compatible values comprise numerical values.

14. The method of optimizing supply networks of claim 12, wherein the numerical values of the preference factors are aggregated to provide an overall optimum priority for each potential resource.

15. The method of optimizing supply networks of claim 14, wherein in allocating the most preferable resource to fulfil the request at least the highest-scoring option is allocated to fulfil the request.

16. The method of optimizing supply networks of claim 15, wherein in allocating the most preferable resource to fulfil the request a plurality of the highest-scoring options are presented to the user, the method further comprising the user choosing which option should be allocated to fulfil the request.

17. The method of optimizing supply networks of claim 1, wherein the method comprises a first initial step of grouping individual resource into supply networks within the database, the individual entries in a supply network sharing one or more resource characteristic.

18. The method of optimizing supply networks of claim 1, wherein the method comprises a second initial step of grouping multiple users into consumer networks within the database, the individual users within a consumer network sharing one or more attributes.

19. The method of optimizing supply networks of claim 1, further comprising assessing the attributes of the user between receiving a resource request from a user and assessing the requirements of the request.

20. The method of optimizing supply networks of claim 1, wherein the method comprises a computer-implemented method.

21. A system for optimizing supply networks comprising a computing system capable of carrying out the method of claim 1.

22. The system for optimizing supply networks of claim 21, comprising:
control and storage hardware configured to act as a database component and a central controller;
communication hardware configured to communicate externally to the system to receive user requests.
